(19) United States
(12) Patent Application Publication
(10) Pub. No.: US 2012/0078088 A1
(43) Pub. Date: Mar. 29, 2012

(54) Medical image projection and tracking system

(75) Inventors: Jennifer J. Whitestone, Germantown, OH (US); Steven H. Mersch, Germantown, OH (US)

(73) Assignee: Point of Contact, LLC.

(21) Appl. No.: 12/927,135

(22) Filed: Nov. 8, 2010

Related U.S. Application Data
(63) Continuation-in-part of application No. 12/924,452, filed on Sep. 28, 2010.

(51) Int. Cl. A61B 5/05

(52) U.S. Cl. 600/425

(57) ABSTRACT

A system comprising a convergent parameter instrument and a laser digital image projector for obtaining a surface map of a target anatomical surface, obtaining images of that surface from a module of the convergent parameter instrument, applying pixel mapping algorithms to impute three dimensional coordinate data from the surface map to a two dimensional image obtained through the convergent parameter instrument, projecting images from the convergent parameter instrument onto the target anatomical surface as a medical reference, and applying a skew correction algorithm to the image.

### CROSS REFERENCE TO RELATED APPLICATIONS

This application claims priority from and is a continuation-in-part of U.S. patent application Ser. No. 12/924,452, entitled “Convergent Parameter Instrument” filed Sep. 28, 2010, which is incorporated herein by reference.

### BACKGROUND OF THE INVENTION

(a) Technical Field

The disclosed system relates to medical imaging and methods for the useful projection of medical images onto a patient's anatomy during, for example, evaluation and/or treatment. More particularly, the system relates to medical imaging and methods for the surface corrected projection of medical images onto a patient's anatomy during evaluation and/or treatment using images obtained in real time and/or reference and/or historical images obtained by medical, photographic, and spectral instruments and/or at least one handheld convergent parameter instrument capable of three dimensional surface imaging, color imaging, perfusion imaging, thermal imaging, and near infrared spectroscopy.

(b) Background of the Invention

Skin, the largest organ of the body, has been essentially ignored in medical imaging. No standard of care regarding skin imaging exists. Computerized Tomography (“CT”), Magnetic Resonance Imaging (“MRI”), and ultrasound are routinely used to image within the body for signs of disease and injury. Researchers and commercial developers continue to advance these imaging technologies to produce improved pictures of internal organs and bony structures. Clinical use of these technologies to diagnose and monitor subsurface tissues is now a standard of care. However, no comparable standard of care exists for imaging skin. Skin assessment has historically relied on visual inspection augmented with digital photographs. Such an assessment does not take advantage of the remarkable advances in nontraditional surface imaging, and lacks the ability to quantify the skin's condition, restricting the clinician's ability to diagnose and monitor skin-related ailments. Electronically and quantitatively recording the skin's condition with different surface imaging techniques will aid in staging skin-related illnesses that affect a number of medical disciplines such as plastic surgery, wound healing, dermatology, endocrinology, oncology, and trauma.

Pressure ulcers are a skin condition with severe patient repercussions and enormous facility costs. Pressure ulcers cost medical establishments in the United States billions of dollars annually. Patients who develop pressure ulcers while hospitalized often increase their length of stay to 2 to 5 times the average. The pressure ulcer, a serious secondary complication for patients with impaired mobility and sensation, develops when a patient stays in one position for too long without shifting their weight. Constant pressure reduces blood flow to the skin, compromising the tissue. A pressure ulcer can develop quickly after a surgery, often starting as a reddened area, but progressing to an open sore and ultimately, a crater in the skin.

Other skin injuries include trauma and burns. Management of patients with severe burns and other trauma is affected by the location, depth, and size of the areas burned, and also affects prediction of mortality, need for isolation, monitoring of clinical performance, comparison of treatments, clinical coding, insurance billing, and medico-legal issues. Current measurement techniques, however, are crude visual estimates for burn location, depth, and size. Depth of the burn in the case of an indeterminate burn is often a “wait and see” approach. Accurate initial determination of burn depth is difficult even for the experienced observer and nearly impossible for the occasional observer. Total Burn Surface Area (“TBSA”) measurements require human input of burn location, severity, extent, and arithmetical calculations, with the obvious risk of human error.

An additional skin ailment is vascular malformation (“VM”). VMs are abnormal clusters of blood vessels that occur during fetal development, but are sometimes not visible until weeks or years after birth. Without treatment, the VM will not diminish or disappear but will proliferate and then involute. Treatment is reserved for life or vision-threatening lesions. A hemangioma may appear to present like a VM. However, it is important to distinguish hemangiomas from the vascular malformations in order to recommend interventions such as lasers, interventional radiology, and surgery. One difference between the hemangioma and vascular malformation can be the growth rate as the hemangiomas grow rapidly compared to the child's growth. Other treatments such as compression garments and drug therapy require a quantitative means of determining efficacy. MRI, ultrasonography, and angiograms are used to visualize these malformations, but are costly and sometimes require anesthesia and dye injections for the patient. A need exists with all skin conditions to enable quantification of changes of the anomalies, to prescribe interventions and determine treatment outcomes.

### SUMMARY

The present disclosure addresses the shortcomings of the prior art and provides a medical imaging and projection system for the surface corrected projection of medical images onto a patient's anatomy during evaluation and/or treatment using images obtained in real time and/or reference and/or historical images obtained by medical, photographic, and spectral instruments and/or at least one handheld convergent parameter instrument capable of three dimensional surface imaging, color imaging, perfusion imaging, thermal imaging, and near infrared spectroscopy. This convergent parameter instrument is a handheld system which brings together a variety of imaging techniques to digitally record parameters relating to skin condition.

The instrument integrates some or all of high resolution color imaging, surface mapping, perfusion imaging, thermal imaging, and Near Infrared (“NIR”) spectral imaging. Digital color photography is employed for color evaluation of skin disorders. Use of surface mapping to accurately measure body surface area and reliably identify wound areas has been proven. Perfusion mapping has been employed to evaluate burn wounds and trauma sites. Thermal imaging is an accepted and efficient technique for studying skin temperature as a tool for medical assessment and diagnosis. NIR spectral imaging may be used to measure skin hydration, an indicator of skin health and an important clue for a wide variety of medical conditions such as kidney disease or diabetes. Visualization of images acquired by the different modalities is controlled through a common control set, such as user-friendly touch screen controls, graphically displayed as 2D and 3D images, separately or integrated, and enhanced using image processing to highlight and extract features. All skin parameter instruments are non-contact which means no additional risk of contamination, infection or discomfort. All scanning modalities may be referenced to the 3D surface acquired by the 3D surface mapping instrument. Combining the technologies creates a multi-parameter system with capability to assess injury to and diseases of the skin.

In one embodiment, the system is a laser digital image projector, a control system to perform target tracking, skew correction, image merging, and pixel mapping coupled with a convergent parameter instrument comprising: a color imaging module, a surface mapping module, a thermal imaging module, a perfusion imaging module, a near infrared spectroscopy module, a common control set for controlling each of the modules, a common display for displaying images acquired by each of the modules, a central processing unit for processing image data acquired by each of the modules, the central processing unit in electronic communication with each of the modules, the common control set, and the common display. The common control set includes an electronic communications interface in embodiments where such functionality is desired.

In another embodiment, the system is laser digital image projector, a control system to perform target tracking, skew correction, image merging, and pixel mapping coupled with a convergent parameter instrument comprising a body incorporating a common display, a common control set, a central processing unit, and between one and four imaging modules selected from the group consisting of: a color imaging module, a surface mapping module, a thermal imaging module, a perfusion imaging module, and a near infrared spectroscopy module. In this embodiment, the central processing unit is in electronic communication with the common display, the common control set, and each of the selected imaging modules, and each of the selected imaging modules are controllable using the common control set, and images acquired by each of the selected imaging modules are viewable on the common display. In this embodiment, the instrument is capable of incorporating at least one additional module from the group into the body, the at least one additional module, once incorporated, being controllable using the common control set and in electronic communication with the central processing unit, and wherein images acquired by the at least one additional module are viewable on the common display.

In a further embodiment, the system is a method for quantitatively assessing an imaging subject's skin, comprising: (a) acquiring at least two skin parameters using a convergent parameter instrument through a combination of at least two imaging techniques, each of the at least two imaging techniques being selected from the group consisting of: (1) acquiring high resolution color image data using a high resolution color imaging module, (2) acquiring surface mapping data using a surface mapping module, (3) acquiring thermal image data using a thermal imaging module, (4) acquiring perfusion image data using a perfusion imaging module, and (5) acquiring hydration data using a near infrared spectroscopy module, (b) using the convergent parameter instrument to select and quantify an imaging subject feature visible in the at least one image, (c) using that imaging subject feature for spatial orientation of current, reference, and historical images and (c) assessing the imaging subject's skin based on the quantified imaging subject feature.

In yet another embodiment, the system is a method for providing a medical reference during patient treatment comprising at least the steps of: (a.) selecting at least one image of a target area either currently acquired from a convergent parameter instrument and/or and image from a reference database, (b.) generating a surface map of a target area, (c) applying an pixel mapping algorithm to infer three dimensional coordinates to two dimensional images based on features of the surface map, (d) applying a skew correction algorithm to compensate for the stretching of a projected two dimensional image across a three dimensional surface and to further adjust for the position of the projector relative to the perspective of the image, and (e) projecting the image(s) onto the target area.

In a further refinement of the preceding embodiment, surgical graphics depicting rescission margins and/or other graphics to assist in a medical procedure can be created and projected alone or in combination with other images.

### BRIEF DESCRIPTION OF THE DRAWINGS

A better understanding of the system will be had upon reference to the following description in conjunction with the accompanying drawings, wherein:

FIG. 1A shows a rear view of an embodiment of a convergent parameter instrument;

FIG. 1B shows a front view of the embodiment of the convergent parameter instrument;

FIG. 1C shows a perspective view of the embodiment of the convergent parameter instrument; and

FIG. 2 shows a schematic diagram of a convergent parameter instrument.

FIG. 3 is a flowchart of a method for using a convergent parameter instrument.

FIG. 4A shows a rear view of an embodiment of a convergent parameter instrument with an integrated laser digital image projector;

FIG. 4B shows a front view of the embodiment of the convergent parameter instrument with an integrated laser digital image projector;

FIG. 5 is a depiction of the use of the laser digital image projector during a surgical procedure.

### DETAILED DESCRIPTION OF THE DISCLOSED EMBODIMENTS

The present disclosure involves the physical and/or system integration of a laser digital image projector 20 with a camera and a source of real time and/or reference and/or historical images, a skew correction algorithm written in machine readable language, a position tracking algorithm written in machine readable language, a pixel mapping algorithm written in machine readable language, and a control system. A convergent parameter instrument 10 can be utilized to supply real time, reference, or historical images for projection onto a patient's anatomy, e.g. area of disease, trauma, or surgical field. Images from other instruments can be uploaded into the control system as can historical images taken of the patient's anatomy in the past and reference images that are not of the patient's anatomy but which may prove useful in education, treatment, or diagnosis.

One imaging technique available from a convergent parameter instrument 10 is high resolution color digital photography, used for the purpose of medical noninvasive optical diagnostics and monitoring of diseases. Digital photography, when combined with controlled solid state lighting, polarization filtering, and coordinated with appropriate image processing techniques, derives more information that the naked eye can discern. Clinically inspecting visible skin color changes by eye is subject to inter and intra-examiner variability. The use of computerized image analysis has therefore been introduced in several fields of medicine in which objective and quantitative measurements of visible changes are required. Applications range from follow-up of dermatological lesions to diagnostic aids and clinical classifications of dermatological lesions. For example, computerized color analysis allows repeated noninvasive quantitative measurements of erythema resulting from a local anesthetic used to inhibit edema and improve circulation in burns.

In one embodiment, the system includes a color imaging module 16, a state of the art, high definition color imaging array, either a complimentary metal oxide semiconductor (“CMOS”) or charge-coupled device (“CCD”) imaging array. The definition of “high resolution” changes as imaging technology improves, but at this time is interpreted as a resolution of at least 5 megapixels. The inventors anticipate using higher resolution imaging arrays as imaging technology improves. The color image can be realized by the use of a Bayer color filter incorporated with the imaging array. In a preferred embodiment, the color image is realized by using sequential red, green, and blue illumination and a black and white imaging array. This preferred technique preserves the highest spatial resolution for each color component while allowing the convergent parameter instrument to select colors which enhances the clinical value of the resulting image. A suitable color imaging module 16 is the Mightex Systems 5 megapixel monochrome CMOS board level array, used in conjunction with sequential red, green, and blue illumination. The color imaging module preferably includes polarization filtering, which removes interfering specular highlights in reflections from wet or glossy tissue, which is common in injured skin, thereby improving the resulting image quality.

Another imaging technique available from a convergent parameter instrument 10 is rapid non-contact surface mapping, used to capture and accurately measure dimensional data on the imaging subject. Various versions of surface mapping exist as commercial products and are either laser-based or structured light scanners, or stereophotogrammetry. Surface mapping has been applied in medicine to measure wound progression, body surface area, scar changes and cranio-facial asymmetry as well as to create orthodontic and other medically-related devices. The availability of three-dimensional data of body surfaces like the face is becoming increasingly important in many medical specialties such as anthropometry, plastic and maxillo-facial surgery, neurosurgery, visceral surgery, and forensics. When used in medicine, surface images assist medical professionals in diagnosis, analysis, treatment monitoring, simulation, and outcome evaluation. Surface mapping is also used for custom orthotic and prosthetic device fabrication. 3D surface data can be registered and fused with 3D CT, MRI, and other medical imaging techniques to provide a comprehensive view of the patient from the outside in.

Examples of the application of surface mapping include the ability to better understand the facial changes in a developing child and to determine if orthodontics influences facial growth. Surface maps from children scanned over time were compared, generating data as absolute mean shell deviations, standard deviations of the errors during shell overlaps, maximum and minimum range maps, histogram plots, and color maps. Growth rates for male and female children were determined, mapped specifically to facial features in order to provide normative data. Another opportunity is the use of body surface mapping as a new alternative for breast volume computation. Quantification of the complex breast region can be helpful in breast surgery, which is shaped by subjective influences. However, there is no generally recognized method for breast volume calculation. Volume calculations from 3D surface scanning have demonstrated a correlation with volumes measured by MRI (r=0.99). Surface mapping is less expensive and faster than MRI, producing the same results. Surface mapping has also been used to quantitatively assess wound-healing rates. As another example, non-contact color surface maps may be used for segmentation and quantification of hypertrophic scarring resulting from burns. The surface data in concert with digital color images presents new insight into the progression and impact of hypertrophic scars.

Included in the system is a surface mapping module 18. Preferably, the surface mapping module 18 offers high spatial resolution and real time operation, is small and lightweight, and has comparatively low power consumption. In one embodiment, the surface mapping module 18 includes an imaging array and a structured light pattern projector 20 spaced apart from the imaging array. In one embodiment, the surface mapping module 18 may be based upon the surface mapping technology developed by Artec Group, Inc., whereby the structured light pattern projector 20 projects a structured pattern of light onto the imaging subject, which is received by the imaging array. Curvature in the imaging subject causes distortions in the received structured light pattern, which may be translated into a three dimensional surface map by appropriate software, as is known in the art. The surface mapping module 18 is capable of imaging surfaces in motion, eliminating any need to stabilize or immobilize an individual or body part of an individual being scanned.

The third imaging technique is digital infrared thermal imaging (“DITI”). DITI is a non-invasive clinical imaging procedure for detecting and monitoring a number of diseases and physical injuries by showing the thermal abnormalities present in the body. It is used as an aid for diagnosis and prognosis, as well as monitoring therapy progress, within many clinical fields, including early breast disease detection, diabetes, arthritis, soft tissue injuries, fibromyalgia, skin cancer, digestive disorders, whiplash, and inflammatory pain. DITI graphically presents soft tissue injury and nerve root involvement, visualizing and recording “pain.” Arthritic disorders generally appear “hot” compared to unaffected areas. Simply recording differences in contralateral regions identifies areas of concern, disease, or injury.

A convergent parameter instrument also includes a thermal imaging module 22. Preferably, the thermal imaging module 22 is small and lightweight, uncooled, and has low power requirements. In one embodiment, the thermal imaging module 22 is microbolometer array. Preferably, the microbolometer array has a sensitivity of 0.1° C. or better. A suitable microbolometer array is a thermal imaging core offered by L-3 Communications Infrared Products.

Perfusion imaging is yet another feature available from a convergent parameter instrument, used to directly measure microcirculatory flow. Commercial laser Doppler scanners, one means of perfusion imaging, have been used in clinical applications that include determining burn injury, rheumatoid arthritis, and the health of post-operative flaps. During the inflammatory response to burn injury, there is an increase in perfusion. Laser Doppler imaging (“LDI”), used to assess perfusion, can distinguish between superficial burns, areas of high perfusion, and deep burns, areas of very low perfusion. Laser Doppler perfusion imaging has also been finding increasing utility in dermatology. LDI has been used to study allergic and irritant contact reactions, to quantify the vasoconstrictive effects of corticosteroids, and to objectively evaluate the severity of psoriasis by measuring the blood flow in psoriatic plaques. It has also been used to study the blood flow in pigmented skin lesions and basal cell carcinoma where it has demonstrated significant variations in the mean perfusion of each type of lesion, offering a noninvasive differential diagnosis of skin tumors 88.

When a diffuse surface such as human skin is illuminated with coherent laser light, a random light interference effect known as a speckle pattern is produced in the image of that surface. If there is movement in the surface, such as capillary blood flow within the skin, the speckles fluctuate in intensity. These fluctuations can be used to provide information about the movement. LDI techniques for blood flow measurements are based on this basic phenomenon. While LDI is becoming a standard, it is limited by specular artifacts, low resolution, and long measurement times.

Included in the system is a perfusion imaging module 24. In one embodiment, the perfusion imaging module 24 is a laser Doppler scanner. In this embodiment, the perfusion imaging module includes a coherent light source 26 to illuminate a surface and at least one imaging array to detect the resulting speckle pattern. In a preferred embodiment, the perfusion imaging module 24 includes a plurality of imaging arrays, each receiving identical spectral content, which sequentially acquire temporally offset images. The differences between these temporally offset images can be analyzed to detect time-dependent speckle fluctuation. A preferred technique for perfusion imaging is described in a co-pending U.S. patent application for a “Perfusion Imaging System” filed by the inventors and incorporated herein by reference.

An additional imaging technique available on a convergent parameter instrument is Near Infrared Spectroscopy (“NIRS”). Skin moisture is a measure of skin health, and can be measured using non-contact NIRS. The level of hydration is one of the significant parameters of healthy skin. The ability to image the level of hydration in skin would provide clinicians a quick insight into the condition of the underlying tissue.

Water has a characteristic optical absorption spectrum in the NIR spectrum. In particular, it includes a distinct absorption band centered at about 1460 nm. Skin hydration can be detected by acquiring a first “data” image of an imaging subject at a wavelength between about 1380-1520 nm, preferably about 1460 nm, and a second “reference” image of an imaging subject at a wavelength less than the 1460 nm absorption band, preferably between about 1100-1300 nm. The first and second images are acquired using an imaging array, such as a NIR sensitive CMOS imaging array. The first and second images are each normalized against stored calibration images of uniform targets taken at corresponding wavelengths. A processor performs a pixel by pixel differencing, either by subtraction or ratio, between the normalized first image and the normalized second image to create a new hydration image. False coloring is added to the hydration image based on the value at each pixel. The hydration image is then displayed to the user on a display. By performing these steps multiple times per second, the user can view skin hydration in real-time or near real-time.

Included in the system is a NIRS module 28. In one embodiment, this module 28 includes an imaging array with NIR sensitivity, and an integrated light source 30 or light filtering means capable of providing near infrared light to the imaging array.

Each of the five imaging techniques produce measurements, numerical values which describe skin parameters such as color, contour, temperature, microcirculatory flow, and hydration. Quantitative determination of these parameters allows quantitative assessment skin maladies, such as, for example, burns, erythema, or skin discoloration, which are normally evaluated only by eye and experience. Each of the imaging techniques in the convergent parameter instrument may be used separately, but additional information may be revealed when images acquired by different techniques are integrated to provide combined images.

Each of the five imaging modules preferably includes a signal transmitting unit, a processor which converts raw data into image files, such as bitmap files. This pre-processing step allows each imaging module to provide the same format of data to the central processing unit (“CPU”) 32, a processor, of the convergent parameter instrument, which reduces the workload of the CPU 32 and simplifies integration of images. The CPU 32 serves to process images, namely, analyzing, quantifying, and manipulating image data acquired by the imaging modules or transferred to the instrument 10.

The surface mapping module 18, NIRS module 28, perfusion imaging module 24, and color imaging module 16 each utilize imaging arrays, such as CMOS arrays. In a preferred embodiment, a given imaging array may be used by more than one module by controlling the illumination of the imaging subject. For example, an imaging array may be used to acquire an image as part of the color imaging module 16 by sequentially illuminating the imaging subject with red, green, and blue light. The same imaging array may later be used to acquire an image as part of the NIRS module 28 by illuminating the imaging subject with light at NIR wavelengths. In this preferred embodiment, fewer imaging arrays would be needed, decreasing the cost of the convergent parameter instrument 10.

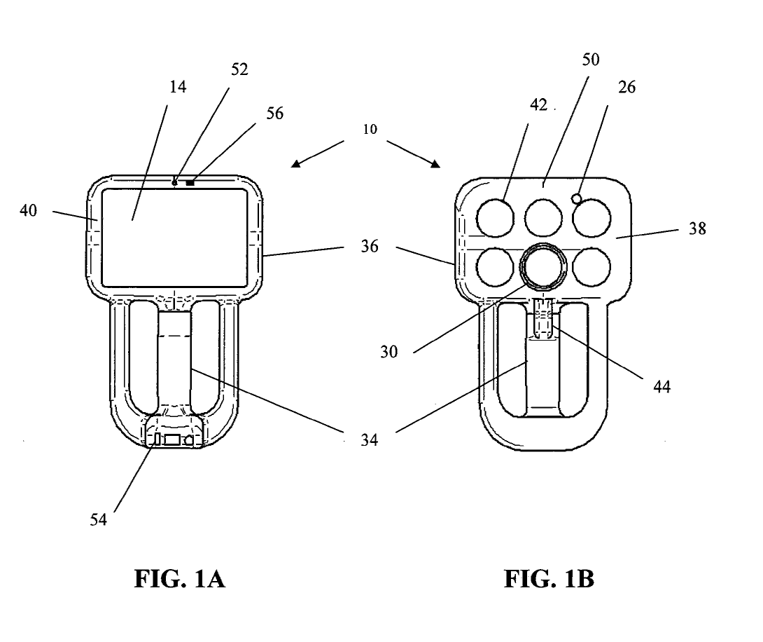

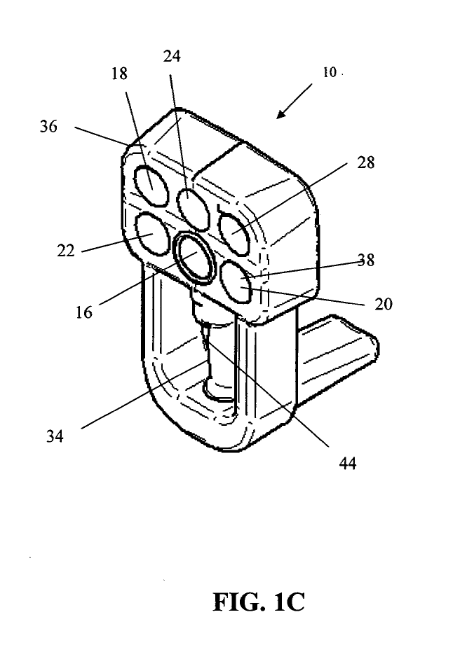

FIGS. 1A, 1B, and 1C depict an embodiment of the system. The convergent parameter instrument 10 is shown comprising a handle 34 attached to a body 36. The body 36 includes a first side 38 and a second side 40. The first side 38 includes one or more apertures 42. In this embodiment, each of the one or more apertures 42 is associated with a single imaging module located within the body 36 and allows electromagnetic radiation to reach the imaging module. In a preferred embodiment, the instrument 10 includes six apertures 42, each associated with one of the five imaging modules described herein (the surface mapping module 18 uses two apertures 42, one for the imaging array and one for the structured light pattern projector 20). In alternate embodiments, the instrument 10 may include a single aperture 42 associated with all imaging modules or any other suitable combination of apertures and modules. For example, in an embodiment where the same imaging array is used with multiple modules, the instrument 10 may include three apertures 42; one for the thermal imaging module 22, one of the structured light pattern projector 20, and one for the imaging arrays which collect color, surface maps, kin hydration, and perfusion data.

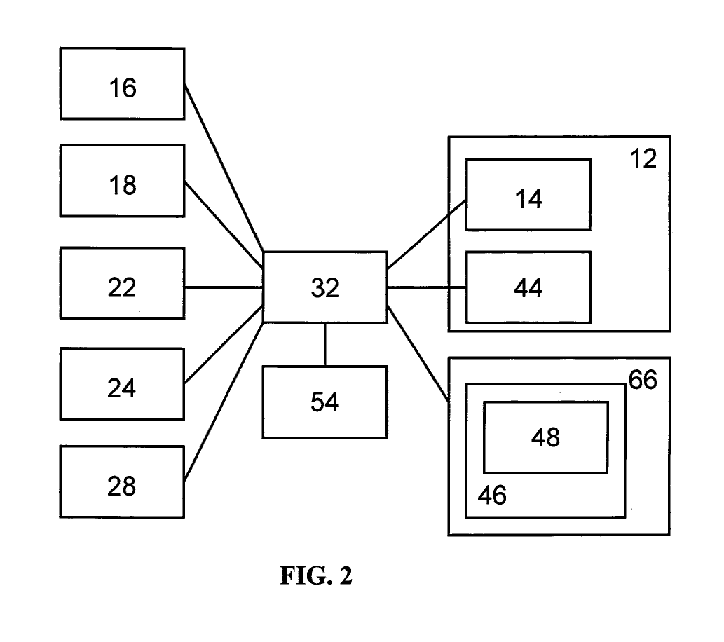

The system includes a common display 14, whereby images acquired by each imaging technique are displayed on the same display 14. The system also includes a common control set 12 (FIG. 2) which controls all imaging modalities and functions of the system. In a preferred embodiment, the common control set 12 includes the display 14, the display 14 being a touch screen display capable of receiving user input, and an actuator 44. In the embodiment displayed in FIGS. 1A, 1B, and 1C, the actuator 44 is a trigger. In other embodiments, the actuator 44 may be a button, switch, toggle, or other control. In the displayed embodiment, the actuator 44 is positioned to be operable by the user while the user holds the handle 34.

The actuator 44 initiates image acquisition for an imaging module. The touch screen display 14 is used to control which imaging module or modules are activated by the actuator 44 and the data gathering parameters for that module or modules. The actuator 44 effectuates image acquisition for all imaging modules, simplifying the use of the instrument 10 for the user. For example, the user may simply select a first imaging technique using the touch screen display 14, and squeeze the actuator 44 to acquire an image using the first imaging module. Alternatively, the user may select first, second, third, fourth, and fifth imaging techniques using the touch screen display 14, and squeeze the actuator 44 a single time to sequentially acquire images using the five modules. The instrument 10 may also provide a real-time or near real-time “current view” of a given imaging module to the user. In one embodiment, this current view is activated by partially depressing the trigger actuator 44. The instrument 10 continuously displays images from a given module, updating the image presented on the display 14 multiple times per second. Preferably, newly acquired images will be displayed 30-60 times per second, and ideally at a frame rate of about 60 times per second, to provide a latency-free viewing experience to the user.

In a preferred embodiment, the instrument 10 is supportable and operable by a single hand of the user. For example, in the embodiment shown in FIGS. 1A, 1B, and 1C, the user's index finger may control the trigger actuator 44 and the user's remaining fingers and thumb grip the handle 34 to support the instrument 10. The user may use his or her other hand to manipulate the touch screen display 14 then, once imaging modules have been selected, preview and acquire images while controlling the instrument with a single hand.

The instrument 10 includes an electronic system for image analysis 46, namely, software integrated into the instrument 10 and run by the CPU 32 which provides the ability to overlay, combine, and integrate images generated by different imaging techniques or imported into the instrument 10. Texture mapping is an established technique to map 2D images (such as the high resolution color images, thermal images, perfusion images, and NIR images) onto the surface of the 3D model acquired using the surface mapping module. This technique allows a user to interact with several forms of data simultaneously. This electronic system for image analysis 46 allows users to acquire, manipulate, register, process, visualize, and manage image data on the handheld instrument 10. Software programs to acquire, manipulate, register, process, visualize, and manage image data are known in the art.

In a preferred embodiment, the electronic system for image analysis 46 includes a database of reference images 48 that is also capable of storing images from the convergent parameter instrument 10 or from an external source. For example, a user of the instrument 10 may compare an acquired image and a reference image using a split screen view on the display 14. The reference image may be a previously acquired image from the same imaging subject, such that the user may evaluate changes in the imaging subject's skin condition over time. The reference image may also be an exemplary image of a particular feature, such as a particular type of skin cancer or severity of burn, such that a user can compare an acquired image of a similar feature on an imaging subject with the reference image to aid in diagnosis. In one embodiment, the user may insert acquired images into the database of reference images 48 for later use.

In one embodiment, the system for image analysis includes a patient positioning system (“PPS”) to aid the comparison of acquired images to a reference image. The user may use the touch screen display 14 to select the PPS prior to acquiring images of the imaging subject. Upon selection of PPS, the user browses through the database of reference images 48 and selects a desired reference image. The display 14 then displays both the selected reference image and the current view of the instrument 10, either in a split screen view or by cycling between the reference image and current view. The user may then position the instrument 10 in relation to the imaging subject to align the current view and reference image. When the user acquires images of the imaging subject, they will be at the same orientation as the reference image, simplifying comparison of the acquired images and the reference image. In one embodiment, the instrument 10 may include image matching software to assist the user in aligning the current view of the imaging subject and the reference image.

The electronic system for image analysis 46 is accessed through the touch screen display 14 and is designed to maximize the value of the portability of the system. Other methods of image analysis include acquiring two images of the same body feature at different dates and comparing the changes in the body feature. Images may be acquired based on a plurality of imaging techniques, the images integrated into a combined image or otherwise manipulated, and reference images provided all on the handheld instrument 10, offering unprecedented mobility in connection with improvements to the accuracy and speed of evaluation of skin maladies. Due to the self-contained, handheld nature of the instrument 10, it is particularly suited to being used to evaluate skin maladies, such as burns, at locations remote from medical facilities. For example, an emergency medical technician could use the instrument 10 to evaluate the severity of a burn at the location of a fire, before the burn victim is taken to a hospital.

The instrument 10 includes light sources according to the requirements of each imaging technique. The instrument 10 includes an integrated, spectrally chosen, stable light source 30, such as a ring of solid state lighting, which includes polarization filtering. In one embodiment, the integrated light source 30 is preferably a circular array of discrete LEDs. This array includes LEDs emitting wavelengths appropriate for color images as well as LEDs emitting wavelengths in the near infrared. Each LED preferably includes a polarization filter appropriate for its wavelength. In another embodiment, the integrated light source 30 may be two separate circular arrays of discrete LEDs, one with LEDs emitting wavelengths appropriate for color imaging and the other with LEDs emitting wavelengths appropriate for NIR imaging. The integrated light source 30, whether embodied in one or two arrays of LEDs, preferably emits in wavelengths ranging from about 400 nm to about 1600 nm. The surface mapping module includes a structured light pattern projector 20 as the light source. Preferably, the structured light pattern projector 20 of the surface mapping module 18 is located at the opposite corner of the body 36 from the imaging array of the surface mapping module 18 to provide the needed base separation required for accurate 3D profiling. A coherent light source 26 is included for the perfusion imaging module 24. Preferably, the coherent light source 26 is a 10 mW laser emitting between about 630-850 nm to illuminate a field of view of about six inches diameter at a distance of about three feet. Thermal imaging requires no additional light source as infrared radiation is provided by the imaging subject. The imaging optics for all imaging modules are designed to provide a similar field of view focused at a common focal distance.

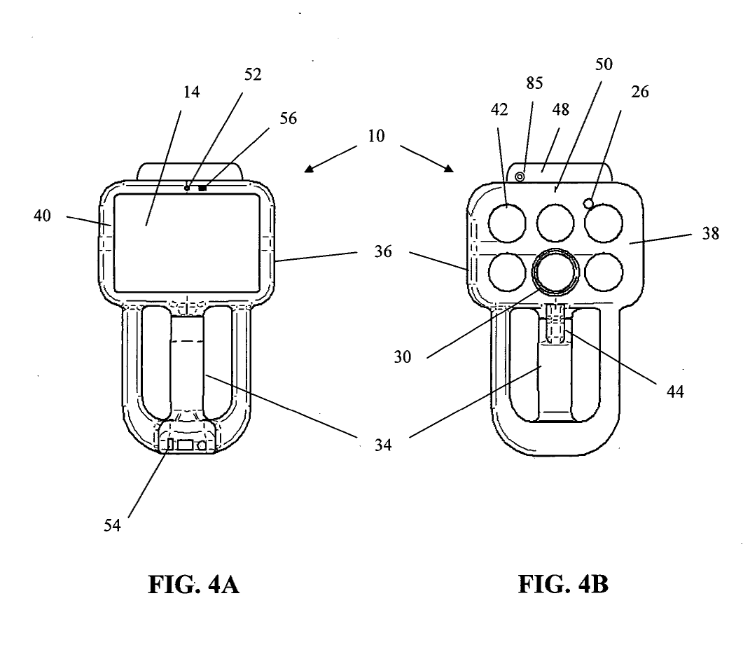

The common field of view and focal distance of the system simplifies image registration and enhances the accuracy of integrated images. In one embodiment, the common focal distance is about three feet. In an additional embodiment, as depicted in FIGS. 4A and 4B, the instrument 10 includes an integrated range sensor 50 and a focus indicator 52 in electronic communication with the range sensor 50. The range sensor 50 is located on the first side 38 of the instrument 10 and the focus indicator 52 is located on the second side 40 of the instrument 10. The range sensor 50 and focus indicator 52 cooperatively determine the range to the imaging subject and signal to the user whether the imaging subject is located at the common focal distance. A suitable range sensor 50 is the Sharp GP2Y0A02YK IR Sensor. In one embodiment, the focus indicator 52 is a red/green/blue LED which emits red when the range sensor 50 detects that the imaging subject is too close, green when the imaging subject is in focus, and blue when the imaging subject is too far.

In an embodiment, as depicted in FIG. 2, the instrument 10 includes data transfer unit 54 for transferring electronic data to and from the instrument 10. The data transfer unit 54 may be used to transfer image data to and from the instrument 10, or introduce software updates or additions to the database of reference images 48. The data transfer unit 54 may be at least one of a USB port, integrated wireless network adapter, Ethernet port, IEEE 1394 interface, serial port, smart card port, or other suitable means for transferring electronic data to and from the instrument 10.

In another embodiment, as depicted in FIG. 4B, the instrument 10 includes an integrated audio recording and reproduction unit 56, such as a combination microphone/speaker. This feature allows the user to record comments to accompany acquired images. This feature may also be used to emit audible cues for the user or replay recorded sounds. In one embodiment, the audio recording and reproduction unit 56 emits an audible cue to the user when data acquisition is complete, indicating that the actuator 44 may be released.

The instrument 10 depicted in FIGS. 1A, 1B, and 1C is only one embodiment of the system. Alternative constructions of the instrument 10 are contemplated which lack a handle 34. In such alternative constructions, the actuator 44 may be located on the body 36 or may be absent and all functions controlled by the touch screen display 14. In other embodiments, the display 14 may not be a touch screen display and may simply serve as an output device. In such embodiments, the common control set 12 would include at least one additional input device, such as, for example, a keyboard. In all embodiments, the instrument 10 is most preferably portable and handheld.

Referring now to FIG. 2, the system includes a CPU 32 in electronic communication with a color imaging module 16, surface mapping module 18, thermal imaging module 22, perfusion imaging module 24, and NIRS imaging module 28. The CPU 32 is also in electronic communication with a common control set 12, computer readable storage media 58, and may receive or convey data via a data transfer unit 54. The common control set 12 comprises the display 14, in its role as a touch screen input device, and actuator 44. The computer readable storage media 58 stores images acquired by the instrument 10, the electronic system for image analysis 46, and image data transferred to the instrument 10.

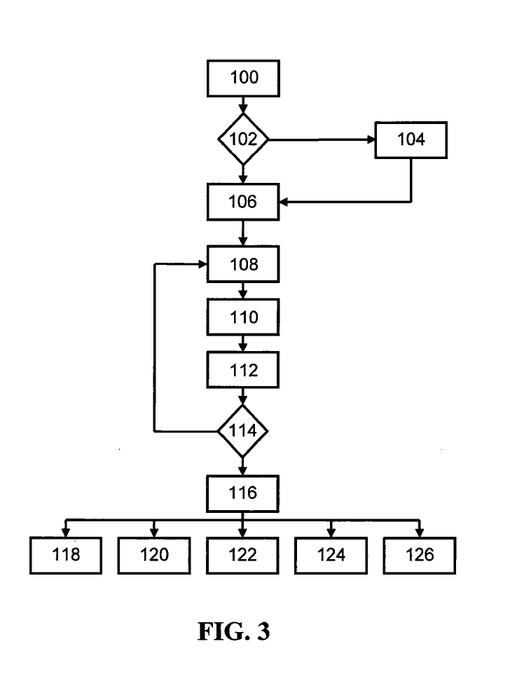

FIG. 3 depicts a method of using a convergent parameter instrument 10. In step 100, a user selects an imaging subject. In step 102, the user. chooses whether to use the PPS. If so, the user selects a reference image from the database of reference images 48 in step 104. In step 106, the user uses the common display 14 to select at least one imaging technique to determine a skin parameter. In step 108, the user orients the instrument 10 in the direction of the imaging subject. In step 110, the user adjusts the distance between the instrument 10 and the imaging subject to place the imaging subject in focus, as indicated by the focus indicator 52. In step 112, where the actuator 44 is a trigger, the user partially depresses the actuator 44 to view the current images of the selected modules on the display 14. The images are presented sequentially at a user programmable rate. In step 114, the user determines whether the current images are acceptable. If the user elected to use the PPS in step 102, the user determines the acceptability of the current images by evaluating whether the current images are aligned with the selected reference image. If the current images are unacceptable, the user returns to step 108. Otherwise, the user fully depresses the actuator 44 to acquire the current images in step 116. Once images are acquired, the user may elect to further interact with the images by proceeding with at least one processing and analysis step. In step 118, the user compares the acquired images to previously acquired images or images in the database of reference images 48. In step 120, the user adds audio commentary to at least one of the acquired images using the audio recording and reproduction unit 56. In step 122, the user stitches, crops, annotates, or otherwise modifies at least one acquired image. In step 124, the user integrates at least two acquired images into a single combined image. In step 126, the user downloads at least one acquired image to removable media or directly to a host computer via the data transfer unit 54.

For an example of the use of the convergent parameter instrument 10, a clinician may wish to document the state of a pressure ulcer on the bottom of a patient's foot and is interested in the skin parameters of color, contour, perfusion, and temperature. The clinician does not desire to use the PPS. Using the touch screen display 14, the clinician selects the color imaging module 16, the surface mapping module 18, the perfusion imaging module 24, and the thermal imaging module 22. The clinician then aims the instrument 10 at the patient's foot, confirms the range is acceptable using the focus indicator 52, and partially depresses the actuator 44. The display 14 then sequentially presents the current views of each selected imaging module in real time. The clinician adjusts the position of the instrument 10 until the most desired view is achieved. The clinician then fully depresses the actuator 44 to acquire the images. Acquisition may require up to several seconds depending on the number of imaging modules selected. Acquired images are stored in computer readable storage media 58, from which they may be reviewed and processed. Processing may occur immediately using the instrument 10 itself or later at a host computer.

In a preferred embodiment, acquired images are stored using the medical imaging standard DICOM format. This format is used with MRI and CT images and allows the user to merge or overlay images acquired using the instrument 10 with images acquired using MRI or CT scans. Images acquired using MRI or CT scans may be input into the instrument 10 for processing using the electronic system for image analysis of the instrument 10. Alternatively, images acquired using the instrument 10 may be output to a host computer and there combined with MRI or CT images.

Although the system is discussed in terms of diagnosis, evaluation, monitoring, and treatment of skin disorders and damage, the system may be used in connection with medical conditions apart from skin or for non-medical purposes. For example, the system may be used in connection with the development and sale of cosmetics, as a customer's skin condition can be quantified and an appropriate cosmetic offered. The system may also be used by a skin chemist developing topical creams or other health or beauty aids, as it would allow quantified determination of the efficacy of the products.

The convergent parameter instrument 10 of the system is modular in nature. The inventors anticipate future improvements in imaging technology for quantifying the five skin parameters. The system is designed such that, for example, a NIRS module 28 based on current technology could be replaced with an appropriately shaped NIRS module 28 of similar or smaller size based on more advanced technology. Each module is in communication with the CPU 32 using a standard electronic communication method, such as a USB connection, such that new modules of the appropriate size and shape may be simply plugged in. Such replacements may require a user to return his or her convergent parameter instrument 10 to the manufacturer for upgrades, although the inventors contemplate adding new modules in the field in future embodiments of the invention. New software can be added to the instrument 10 using the data transfer unit 54 to allow the instrument 10 to recognize and control new or upgraded modules.

When used for certain purposes, not all five imaging modules may be necessary to perform the functions desired by the user. In one embodiment, the instrument 10 may include less than five imaging modules, such as a least one imaging module, at least two imaging modules, at least three imaging modules, or at least four imaging modules. Any combination of imaging modules may be included, based on the needs of the user. A user may purchase an embodiment of the system including less than all five of the described imaging modules, and have at least one additional module incorporated into the body 36 of the instrument 10 at a later time. The modular design of the instrument 10 allows for additional modules to be controllable by the common control set 12 and images acquired using the additional modules to viewable on the common display 14.

When utilized to project a reference or captured image onto an anatomical field, a 3D camera integrated or in communication with a convergent parameter instrument can collect a 3D framework image. A projector 20 projects a structured light pattern onto the field and at least one camera takes an image which is subsequently rasterized. Changes in the structured light pattern are translated into 3D surface data by employing triangulation methodology between the imaging axis and pattern projection. The imager subsequently collects a color image which is then integrated onto the 3D framework, using the 3D surface data as a template for the correction of images applied to the 3D surface. In an alternative embodiment, as depicted in FIG. 4, the laser digital image projector 20 can be integrated with a convergent parameter instrument 10. The “lens” 85 of the integrated laser digital image projector 20 is positioned facing the first side 38 of the convergent parameter device 10.

Various embodiments employ a tracking and alignment system with the projected images. Virtual characterization can be accomplished by associating the features of an image with 3D data with a 2D image. When the projected 2D image is directed onto a 3D surface, skewing of that projected image will inevitably occur on the 3D surface. Image correction techniques are utilized to compensate for the skewing of the projected image across a 3D surface and, depending on the contours of the anatomical surface, results in alignment of the prominent features of the image onto the prominent features of the imaged anatomical target. Image correction can employ a technique known as “keystoning” to alter the image depending on the angle of the projector 20 to the screen, and the beam angle, when the surface is substantially flat, but angled away from the projector 20 on at least one end. As the surface geometry changes, the angle of the projector 20 to the anatomical surface also changes. Stereo imaging is useful since two lenses are used to view the same subject image, each from a slightly different perspective, thus allowing a three dimensional view of the anatomical target. If the two images are not exactly parallel, this causes a keystone effect.

The pixel center-point and/or vertices of each pixel of the color image may be associated with a coordinate in 3D space located on the surface of the established 3D framework. Perspective correct texturing is one useful method for interpolating 3D coordinates of rasterized images. Another method of interpolation includes perspective correct texture mapping is a form of texture coordinate interpolation where the distance of the pixel from the viewer is considered as part of the texture coordinate interpolation. Texture coordinate wrapping is yet another methodology used to interpolate texture coordinates. In general, texture coordinates are interpolated as if the texture map is planar. The map coordinate is interpolated as if the texture map is a cylinder where 0 and 1 are coincidental. Texture coordinate wrapping may be enabled for each set of texture coordinates, and independently for each coordinate in a set. With planar interpolation, the texture is treated as a 2-D plane, interpolating new texels by taking the shortest route from point A within a texture to point B.

At least structured light pattern projector 20 is a pico laser image projector 20, such as the type available from Microvision, Inc., is positioned within the imager system at an optical axis similar to but necessarily different than the color imager or 3D imager. Using the global coordinate system of the imager, a map is created to associate 3D coordinates with the projected 3D coordinates and related pixel properties, e.g. color, {X_{1 . . . n}, Y_{1 . . . n}, Y_{1 . . . n}), and C(X_{1 . . . n}, Y_{1 . . . n}} where X_{1 . . . n}, Y_{1 . . . n} are the 2D array of pixels that the pico projector 20 can project, Zn is the distance to the surface for pixel (Xn, Yn), and Cn is an assigned property for pixel (Xn, Yn) such as color.

Using triangulation between the position of the pico projector 20 and the global coordinate system of the imager, the projected pixel (Xn, Yn, Zn, Cn) strikes the real surface at the corresponding image's virtual image location and illuminates the surface at this location with the appropriate color. The pico laser projector 20 inherently has the ability to project clearly on any surface without focusing via optics thus is optimal for projecting on a 3D surface and currently has the processing capacity to refresh approximately 30 times per second.

A skew correction algorithm modifies the projected two dimensional image to compensate for skewing related to the spatial orientation of the digital image projector 20 relative to a surface onto which the two dimensional image is projected. Associating the pixels of a prominent surface feature or artificial reference point with the same target in a projected image provides a indication of the amount of skewing and permits corrective best fit measures to be applied to realign the images in various embodiments to provide a perspective accurate image.

A further embodiment of the skew correction algorithm compensates for the distance of the projector 20 from the target surface and adjusts the projected image accordingly so as to project an appropriate size image to overlay on the target surface. The use of a sizing reference point such as a target surface feature or artificial reference can optionally be used in various embodiments whereby the image is resized to match the sizing reference point. Alternatively the distance can be an input into the control system. Additionally, the projector 20 may be somewhat mobile so as to facilitate its repositioning, thus permitting a manual resizing of the image.

The control system processes images collected from the convergent parameter instrument or other imaging device, including projected images on a 3D surface such as an anatomical surface, and tracks movement of the surface by comparing and contrasting differences between reference lines and or structures on the 3D surface with the projected image from the pico projector 20. The control system then modifies the projected image to optimize the overlay from the projected image to current 3D surface orientation and topography by recharacterizing the 3D framework. The use of multiple projectors 20 is warranted when shadows become an issue, when larger portions of the 3D surface need to be projected, or whenever projection from multiple angles is required. Alternatively, the use of multiple projectors 20 can be combined with the use of multiple convergent parameter instruments or other imagers.

In one embodiment, the convergent parameter instrument 10, when used in a patient care setting, provides real-time diagnostics and feedback during treatment by utilizing a pico projector 20 as a laser digital image projector 20 to project processed images, e.g. surface and/or subsurface images acquired by the convergent parameter instrument or other device such as an x-ray, CT Scan, or MRI, onto the tissue or organs being imaged for real-time use by the health care provider. Images can be projected in real-time and/or from a reference set. Images can also be modified by the user to include artifacts such as excision margins. The image is collected, processed, and projected in a short enough time period so as to make the image useful and relevant to the health care provider when projected. Useful applications include visualization of surface and subsurface skin conditions and afflictions, e.g. cancer, UV damage, thermal damage, radiation damage, hydration levels, collagen content and the onset of ulcers as well as the evaluation of lesions, psoriasis and icthyosis.

Subsurface skin tumors present themselves as objects with markedly different properties relative to the surrounding healthy tissue. The displacement of fibrillar papillary dermis by the softer, cellular mass of a growing melanoma is one such example. Optical elastographic techniques may provide a means by which to probe these masses to determine their state of progression and thereby help to determine a proper means of disease management. Other skin afflictions, such as psoriasis, previously discussed, and icthyosis, also present as localized tissue areas with distinct physical properties that can be characterized optically.

An additional application includes the delineation between zones of damaged tissue and healthy tissue for use in treatment and education. Perfusion is one example of the usefulness of projected delineation. Reduced arterial blood flow causes decreased nutrition and oxygenation at the cellular level. Decreased tissue perfusion can be transient with few or minimal consequences to the health of the patient. If the decreased perfusion is acute and protracted, it can have devastating effects on the patient's health. Diminished tissue perfusion, which is chronic in nature, invariably results in tissue or organ damage or death.

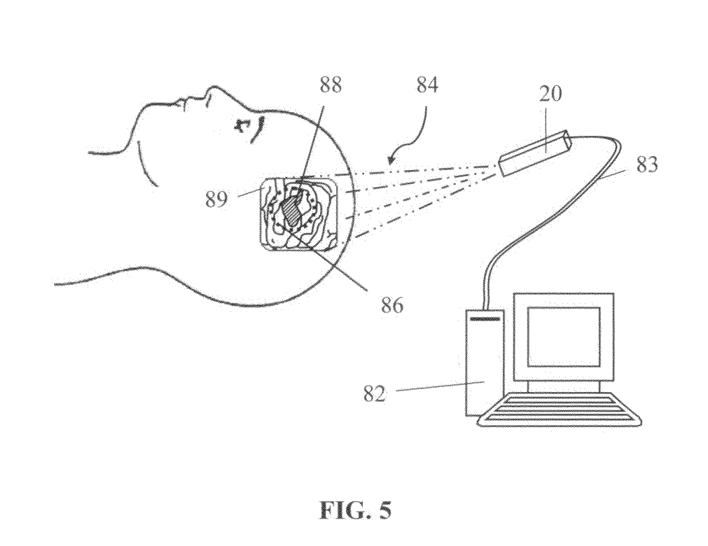

As shown in FIG. 5, delineation by projected image is useful to optimize excision and/or resection margins 86. In the depicted embodiment, a control system 82 functions to control a laser digital image projector 20 through a wired connection 83. A structured light pattern 84 is projected onto an anatomical target 89 to graphically indicate a rescission margin 86, i.e. zone of rescission 86 around a tumor 88. There is no accepted standard for the quantity of healthy or viable tissue to be removed and the effect of positive margins on recurrence rate in malignant tumors 88 appears to be considerably dependent on the site of the tumor 88. The extent of tumor 88 volume resection is determined by the need for cancer control and the peri-operative, functional and aesthetic morbidity of the surgery.

Resection margins 86 are presently assessed intra-operatively by frozen section and retrospectively after definitive histological analysis of the resection specimen. There are limitations to this assessment. The margin 86 may not be consistent in three dimensions and may be susceptible to errors in sampling and histological interpretation. Determining the true excision margin 86 can be difficult due to post-excision changes from shrinkage and fixation.

The use of large negative margins 86 unnecessarily removes too much healthy tissue and close or positive margins increases the risk of failing to remove foreign matter or enough of the target tissue, e.g. tissue that is cancerous or otherwise nonviable or undesirable. Negative margins 86 that remove as little healthy or viable tissue as possible while minimizing the risk of having to perform additional surgery are desirable.

In yet another embodiment, the convergent parameter instrument provides reference images from a database 48 for projection to provide guides for incisions, injections, or other invasive procedures, with color selection to provide contrast with the tissue receiving the projection. Useful applications include comparing and contrasting the progression of healing, visualizing subsurface tissue damage or structures including vasculature and ganglia.

The foregoing detailed description is given primarily for clearness of understanding and no unnecessary limitations are to be understood therefrom for modifications can be made by those skilled in the art upon reading this disclosure and may be made without departing from the spirit of the invention and scope of the appended claims.

What is claimed is:

1. A medical image projection system comprising:
an imaging system, wherein said imaging system is capable of generating a surface map in three dimensions for an imaged surface and communicating said surface map as surface map data;
a control system having at least one associated computer readable storage media capable of storing instructions written in a machine readable language, a user interface, a display, an electronic communications interface, a means for processing data, a means for receiving said surface map data from said imaging system, and a means for executing instructions written in said machine readable language;
a pixel mapping algorithm for producing a dimension adjusted image, wherein a pixel mapping set of instructions written in said machine readable language associates three dimensional coordinates related at least in part to said surface map data with pixels obtained from a two dimensional image of said imaged surface;
a skew correction algorithm for producing a skew adjusted image, wherein instructions written in said machine readable language modify said two dimensional image pixel positions so as to minimize the visual skewing of said two dimensional image across said imaged surface and
a laser digital image projector in electronic communication with said control system for projecting an image modified by said pixel mapping algorithm and said skew correction algorithm onto said imaged surface.

2. The medical image projection system of claim 1, wherein said skew correction algorithm modifies said two dimensional image to compensate for skewing related to the spatial orientation of said digital image projector relative to a surface onto which said two dimensional image is projected by said laser digital image projector.

3. The medical image projection system of claim 1, wherein said skew correction algorithm modifies said two dimensional image to compensate for the distance of said digital image projector relative to said imaged surface onto which said two dimensional image is projected by said laser digital image projector.

4. The medical image projection system of claim 1, wherein said pixel mapping algorithm associates the pixels of said two dimensional image to coordinates across said imaged surface by associating a surface feature for which position data is available in three dimensions with said surface feature identifiable in said two dimensional image and finding a best fit for the two images through modification of pixel coordinate data for said two dimensional image to ensure a perspective accurate representation of said two dimensional image when projected on said imaged surface by said laser digital image projector.

5. The medical image projection system of claim 4, further comprising a position tracking algorithm, wherein position tracking instructions are written in said machine readable language whereby a structured combination of pixels of a previously obtained image is associated with a feature of a tracked surface and their relative positions are utilized to adjust the projected image to substantially mimic changed viewing and projection perspectives.

6. The medical image projection system of claim 1, wherein said two dimensional image is obtained through a Convergent Parameter instrument having at least two modules selected from the group consisting of a color imaging module, a surface mapping module, a thermal imaging module, a perfusion imaging module, and a near infrared spectroscopy module.

7. The medical image projection system of claim 6, wherein said surface map is obtained from said Convergent Parameter instrument.

8. The medical image projection system of claim 6, wherein said digital laser image projector is integrated with said Convergent Parameter instrument.

9. The medical image projection system of claim 6, wherein said at least one associated computer readable storage media and said control system are integrated with said Convergent Parameter instrument.

10. The medical image projection system of claim 9, further comprising a data transfer unit.

11. The medical image projection system of claim 1, wherein said control system is configured to combine a plurality of images for simultaneous projection by said digital laser image projector.

12. The medical image projection system of claim 11, further comprising a database capable of storing images.

13. The medical image projection system of claim 12, wherein said database capable of storing images contains stored images selected from the group consisting of reference medical images, patient historical images, and current images.

14. A medical image projection system comprising:
an imaging system comprised of a Convergent Parameter instrument having at least two modules selected from the group consisting of a color imaging module, a surface mapping module, a thermal imaging module, a perfusion imaging module, and a near infrared spectroscopy module, wherein said imaging system is capable of generating a surface map in three dimensions for an imaged surface and communicating said surface map as surface map data;
a control system having at least one associated computer readable storage media capable of storing instructions written in a machine readable language a user interface, a display, an electronic communications interface, a means for processing data, a means for receiving said surface map data from said imaging system, and a means for executing instructions written in said machine readable language;
a pixel mapping algorithm for producing a dimension adjusted image, wherein a pixel mapping set of instructions written in said machine readable language associates three dimensional coordinates related at least in part to said surface map data with pixels obtained from a two dimensional image of said imaged surface;
a skew correction algorithm for producing a skew adjusted image, wherein instructions written in said machine readable language modify said two dimensional image pixel positions so as to minimize the visual skewing of said two dimensional image across said imaged surface and
a laser digital image projector in electronic communication with said control system for projecting an image modified by said pixel mapping algorithm and said skew correction algorithm onto said imaged surface.

15. The medical image projection system of claim 14, wherein said skew correction algorithm modifies said two dimensional image to compensate for skewing related to the spatial orientation of said digital image projector relative to a surface onto which said two dimensional image is projected.

16. The medical image projection system of claim 14, wherein said skew correction algorithm modifies said two dimensional image to compensate for the distance of said digital image projector relative to said imaged surface onto which said two dimensional image is projected.

17. The medical image projection system of claim 14, wherein said pixel mapping algorithm associates the pixels of said two dimensional image to coordinates across said imaged surface by associating a surface feature for which position data is available in three dimensions with said surface feature identifiable in said two dimensional image and finding a best fit for the two images through modification of coordinate data from said two dimensional image.

18. The medical image projection system of claim 14, further comprising a position tracking algorithm, wherein position tracking instructions are written in said machine readable language whereby a structured combination of pixels of a previously obtained image is associated with a feature of a tracked surface and their relative positions are utilized to adjust the projected image to substantially mimic changed viewing and projection perspectives.

19. The medical image projection system of claim 14, wherein said control system is configured to combine a plurality of images for simultaneous projection by said digital laser image projector.

20. The medical image projection system of claim 14, wherein said at least one associated computer readable storage media and said control system are integrated with said Convergent Parameter instrument.

21. The medical image projection system of claim 20, further comprising a data transfer unit.

22. The medical image projection system of claim 21, further comprising a database capable of storing images.

23. The medical image projection system of claim 22, wherein said database capable of storing images contains stored images selected from the group consisting of reference medical images, patient historical images, and current images.

24. A medical image projection system comprising:
an imaging system comprised of a Convergent Parameter instrument having at least two modules selected from the group consisting of a color imaging module, a surface mapping module, a thermal imaging module, a perfusion imaging module, and a near infrared spectroscopy module, wherein said imaging system is capable of generating a surface map in three dimensions for an imaged surface and communicating said surface map as surface map data;
a control system having at least one associated computer readable storage media capable of storing instructions written in a machine readable language a user interface, a display, an electronic communications interface, a means for processing data, a means for receiving said surface map data from said imaging system, and a means for executing instructions written in said machine readable language;
a pixel mapping algorithm for producing a dimension adjusted image, wherein a pixel mapping set of instructions written in said machine readable language associates three dimensional coordinates related at least in part to said surface map data with pixels obtained from a two dimensional image of said imaged surface;
a skew correction algorithm for producing a skew adjusted image, wherein instructions written in said machine readable language modify said two dimensional image pixel positions so as to minimize the visual skewing of said two dimensional image across said imaged surface, correct the projected image the spatial orientation of said digital image projector relative to a surface onto which said two dimensional image is projected, and the distance of said digital image projector relative to said imaged surface onto which said two dimensional image is projected; and
a laser digital image projector in electronic communication with said control system for projecting an image modified by said pixel mapping algorithm and said skew correction algorithm onto said imaged surface.

25. The medical image projection system of claim 24, wherein said pixel mapping algorithm associates the pixels of said two dimensional image to coordinates across said imaged surface by associating a surface feature for which position data is available in three dimensions with said surface feature identifiable in said two dimensional image and finding a best fit for the two images through modification of coordinate data from said two dimensional image.

26. The medical image projection system of claim 24, further comprising a position tracking algorithm, wherein position tracking instructions are written in said machine readable language whereby a structured combination of pixels of a previously obtained image is associated with a feature of a tracked surface and their relative positions are utilized to adjust the projected image to substantially mimic changed viewing and projection perspectives.

27. The medical image projection system of claim 24, wherein said control system is configured to combine a plurality of images for simultaneous projection by said laser digital image projector.

28. The medical image projection system of claim 24, wherein said at least one associated computer readable storage media and said control system are integrated with said Convergent Parameter instrument.

29. The medical image projection system of claim 24, further comprising a data transfer unit.

30. The medical image projection system of claim 24, further comprising a database capable of storing images.

31. The medical image projection system of claim 30, wherein said database capable of storing images contains stored images selected from the group consisting of reference medical images, patient historical images, and current images.
